(19) United States
(12) Patent Application Publication
(10) Pub. No.: US 2024/0225592 A1
(43) Pub. Date: Jul. 11, 2024

(54) SENSOR ASSEMBLY WITH SET ACOUSTIC MATCHING LAYER THICKNESS FOR INTRALUMINAL SENSING DEVICE

(71) Applicant: PHILIPS IMAGE GUIDED THERAPY CORPORATION, SAN DIEGO, CA (US)

(72) Inventors: Justin Patrick MAY, RANCHO MISSION VIEJO, CA (US); Ramiro REYES, SAN DIEGO, CA (US)

(21) Appl. No.: 18/562,878

(22) PCT Filed: Jun. 7, 2022
(86) PCT No.: PCT/EP2022/065310

§ 371 Date: Nov. 21, 2023

Related U.S. Application Data
(60) Provisional application No. 63/197,960, filed on Jun. 7, 2021.

(51) Int. Cl. A61B 8/12; A61B 5/00; A61B 8/00; A61B 8/06

(52) U.S. Cl. CPC A61B 8/12; A61B 5/6851; A61B 8/06; A61B 8/445; A61B 8/4494

(57) ABSTRACT

An intraluminal sensing device may include an elongate member, a sensor, an acoustic matching layer, and a housing. The elongate member may be configured to be positioned within a body lumen of a patient. The sensor may be configured to obtain physiological data while positioned within the body lumen and may include a proximal surface and an opposite, distal surface. The acoustic matching layer may be disposed on the distal surface. The housing may be positioned at a distal portion of the elongate member and may terminate at a distal end. The housing may include a hollow interior with a planar surface, and the sensor may be positioned within the hollow interior such that the proximal surface of the sensor is disposed on the planar surface. A thickness of the acoustic matching layer may be defined by a distance between the distal surface and the distal end.

### TECHNICAL FIELD

The present disclosure relates generally to intraluminal sensing devices and, in particular, to intraluminal sensing devices comprising a sensor with an acoustic matching layer on a surface of the sensor positioned within a housing. More specifically, the housing may be configured such that a thickness of the acoustic matching layer is defined by a distance between a distal surface of the sensor and a distal end of the housing.

### BACKGROUND

Intraluminal sensing devices, such as intravascular sensing devices, may include a sensor configured to obtain physiological data while positioned within a lumen, such as a blood vessel. For instance, such devices may include an imaging apparatus, a flow sensor, or a pressure sensor sized and shaped to be positioned within the lumen and configured to capture images, flow data, or pressure data within the lumen. In some cases, an acoustic matching layer may be applied to such a sensor. Properties of the acoustic matching layer, including dimensions of the acoustic matching layer, may impact a performance (e.g., an accuracy, a precision, and/or resolution of data) of the sensor. As such, variations in the dimensions of the acoustic matching layer across different intraluminal sensing devices may result in unreliable or inconsistent performance of the devices.

### SUMMARY

Disclosed herein is an intraluminal sensing device (e.g., an intravascular sensing device) that may be configured to obtain physiological data while positioned within a lumen, such as a blood vessel. The device includes a flexible elongate member (e.g., a guidewire and/or a catheter), a housing, and a sensor (e.g., a sensing component), which may be configured to obtain the physiological data and may be positioned within the housing. The sensor may include a proximal surface and an opposite, distal surface, as well as one or more electrical and/or electronic components, such as an ultrasound transducer. Further an acoustic matching layer may be positioned on at least the distal surface of the sensor. A thickness of the acoustic matching layer may be defined (e.g., set) by a distance between the distal surface and a distal end of the housing, where the housing terminates. For instance, the housing may include a hollow interior with a planar surface, such as a hollow interior defined by a counterbore, and the sensor may be positioned within the housing such that the proximal surface of the sensor is positioned on the planar surface of the housing. To that end, the sensor may be positioned within a portion of the housing such that the sensor is positioned between planar surface and the distal end of the housing and spaced from the distal end by the distance defining the thickness of the acoustic matching layer. In this regard, a distal end of the acoustic matching layer may be flush (e.g., coplanar) with the distal end of the housing. More specifically, the distal end of the housing may serve as a visual and/or a physical guide for applying the acoustic matching or adjusting an applied acoustic matching layer to have the defined thickness. In this way, the thickness of the acoustic matching layer may be defined on components (e.g., the sensor and the housing) with dimensions having a fixed relationship, which may ensure desired dimensioning of the acoustic matching layer is achieved and may reduce or prevent inconsistencies in performance of the sensor that may otherwise be caused by inconsistent or incorrect acoustic matching layer dimensions.

In an exemplary aspect, an intraluminal sensing device includes a flexible elongate member including a distal portion and a proximal portion and configured to be positioned within a body lumen of a patient. The intraluminal sensing device may further include a sensor configured to obtain physiological data while positioned within the body lumen. The sensor may include a proximal surface and an opposite, distal surface. The intraluminal sensing device may also include an acoustic matching layer disposed on the distal surface of the sensor and a housing positioned at the distal portion of the flexible elongate member and terminating at a distal end. The housing may include a hollow interior with a planar surface. The sensor may be positioned within the hollow interior of the housing such that the proximal surface of the sensor is disposed on the planar surface of the hollow interior, and a thickness of the acoustic matching layer may be defined by a distance between the distal surface of the sensor and the distal end of the housing.

In some aspects, a distal end of the acoustic matching layer is flush with the distal end of the housing. In some aspects, the acoustic matching layer includes an adhesive. In some aspects, the sensor further includes a side surface, and the adhesive may be disposed on the side surface of the sensor. In some aspects, the proximal surface of the sensor is planar, and the sensor is positioned within the housing such that the proximal surface is parallel with the planar surface along the entire planar surface. In some aspects, the hollow interior may include a counterbore. The counterbore may include a thru-hole extending through the planar surface. In some aspects, at least a portion of the thru-hole is proximal of the planar surface. In some aspects, the housing includes a plurality of layers formed atop one another such that the plurality of layers defines a continuous surface. Further, the hollow interior may be defined by the continuous surface. In some aspects, the sensor further includes an insulating layer. In some aspects, the acoustic matching layer is disposed on the insulating layer. In some aspects, the insulating layer may include a first material and the acoustic matching layer may include a different, second material. In some aspects, the sensor includes a flow sensor. In some aspects, the intraluminal sensing device, further includes a wire assembly coupled to the sensor and extending through a portion of the hollow interior proximal of the planar surface. In some aspects, the intraluminal sensing device, further includes an adhesive positioned between the sensor and the housing and configured to secure the sensor to the housing. In some aspects, the adhesive includes a first material and the acoustic matching layer includes a different, second material.

In an exemplary aspect, an intravascular flow-sensing device includes a guidewire. The guidewire may include a distal portion and a proximal portion and may be configured to be positioned within a blood vessel of a patient. The intravascular flow-sensing device may further include a flow sensor configured to obtain intravascular flow data while positioned within the blood vessel. The flow sensor may include a proximal surface and an opposite, distal surface. The intravascular flow-sensing device may further include an acoustic matching layer disposed on a distal surface of the flow sensor and a housing positioned at the distal portion of the guidewire and terminating at a distal end. The housing may include a hollow interior defined by a counterbore. The counterbore may include a planar surface and a thru-hole. The flow sensor may be positioned within the housing such that the proximal surface of the flow sensor is disposed on the planar surface of the counterbore and a thickness of the acoustic matching layer is defined by a distance between the distal surface of the flow sensor and the distal end of the housing.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic side view of an intravascular sensing system that includes an intravascular device, in accordance with at least one embodiment of the present disclosure.

FIG. 2 is a diagrammatic cross-sectional side view of a sensor assembly, in accordance with at least one embodiment of the present disclosure.

FIG. 3 is a flow chart of a method for assembling a sensor assembly, in accordance with at least one embodiment of the present disclosure.

FIG. 4 is a diagrammatic cross-sectional side view of a sensor sub-assembly, in accordance with at least one embodiment of the present disclosure.

FIG. 5 is a diagrammatic cross-sectional side view of a sensor sub-assembly positioned within a housing, in accordance with at least one embodiment of the present disclosure.

FIG. 6 is a flow chart of a method for assembling a sensor assembly, in accordance with at least one embodiment of the present disclosure.

FIG. 7 is a diagrammatic cross-sectional side view of a sensor sub-assembly positioned within a housing, in accordance with at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

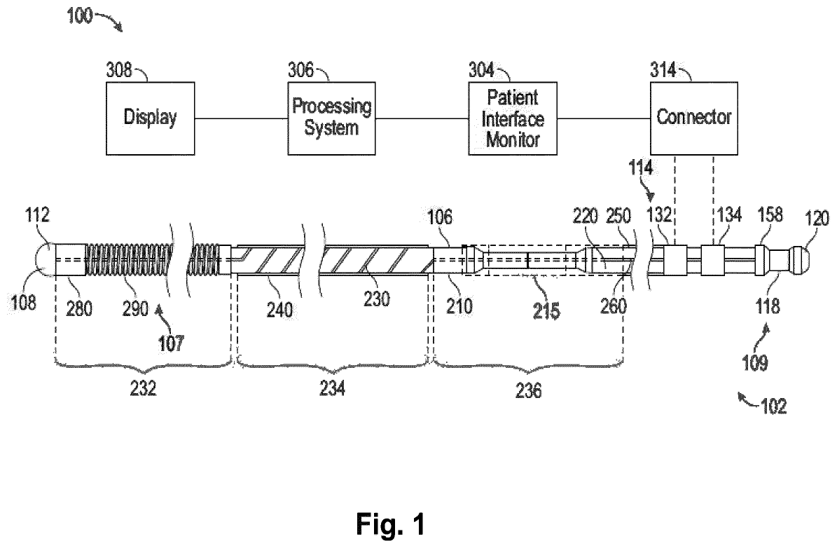

FIG. 1 is a diagrammatic side view of an intravascular sensing system 100 (e.g., an intraluminal sensing system) that includes an intravascular device 102 (e.g., an intraluminal sensing device) comprising a sensing component 112 positioned within a housing 280 that includes a conductive material and a non-conductive material, according to aspects of the present disclosure. The intravascular device 102 can be an intravascular guidewire sized and shaped for positioning within a vessel of a patient. In some embodiments, the intravascular device 102 can be or can interface with a catheter sized and shaped for positioning within a vessel of a patient. The intravascular device 102 can include a distal tip 108 and a sensing component 112. The sensing component 112 can be an electronic, electromechanical, mechanical, optical, and/or other suitable type of sensor. For example, the sensing component 112 can be a flow sensor configured to measure the velocity of blood flow within a blood vessel of a patient, a pressure sensor configured to measure a pressure of blood flowing within the vessel, or another type of sensor including but not limited to a temperature or imaging sensor. For example, flow data obtained by a flow sensor can be used to calculate physiological variables such as coronary flow reserve (CFR). Pressure data obtained by a pressure sensor may for example be used to calculate a physiological pressure ratio (e.g., FFR, iFR. Pd/Pa, or any other suitable pressure ratio). An imaging sensor may include an intravascular ultrasound (IVUS), intracardiac echocardiography (ICE), optical coherence tomography (OCT), or intravascular photoacoustic (IVPA) imaging sensor.

In some embodiments, the sensing component 112 may include one or more transducers, such as one or more ultrasound transducer elements. The one or more ultrasound transducer element (e.g., an acoustic element) may be configured to emit ultrasound energy and receive echoes corresponding to the emitted ultrasound energy. Further, the one or more ultrasound transducer elements may include a piezoelectric/piezoresistive element, a piezoelectric micromachined ultrasound transducer (PMUT) element, a capacitive micromachined ultrasound transducer (CMUT) element, and/or any other suitable type of ultrasound transducer element. The one or more ultrasound transducer elements may further be in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can include a microbeamformer (pBF). In other embodiments, one or more of the ICs includes a multiplexer circuit (MUX).

Further the one or more transducers of the sensing component 112 may be arranged in any suitable configuration. For example, an imaging sensor can an array of ultrasound transducer elements, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The an array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

In an exemplary embodiment, the sensing is a flow sensor, which includes a single ultrasound transducer element, such as the transducer elements described above. The transducer element emits ultrasound signals and receives ultrasound echoes reflected from anatomy (e.g., flowing fluid, such as blood). The transducer element generates electrical signals representative of the echoes. The signal-carrying filars carry this electrical signal from the sensor at the distal portion to the connector at the proximal portion. The processing system processes the electrical signals to extract the flow velocity of the fluid. In other embodiments, the device 102 may be used to examine any number of anatomical locations and tissue types, including without limitation, organs including the liver, heart, kidneys, gall bladder, pancreas, lungs: ducts: intestines: nervous system structures including the brain, dural sac, spinal cord and peripheral nerves: the urinary tract: as well as valves within the blood, chambers or other parts of the heart, and/or other systems of the body. In addition to natural structures, the device 102 may be used to examine man-made structures such as, but without limitation, heart valves, stents, shunts, filters and other devices. In some embodiments, the sensing component 112 may include an imaging component (e.g., an intravascular ultrasound imaging component), a measurement component (e.g., a pressure, flow; or temperature sensor) and/or a treatment component (e.g., an ablation component).

In some embodiments the sensing component 112 may be fully or partially enclosed within a housing 280. In some embodiments, the sensing component 112 is located at or near the distal end of a flexible elongate member, and may include a distal tip 108 (e.g., an atraumatic tip). In some embodiments, one or more electronic components, such as the sensing component 112, can be located at the distal portion of the flexible elongate member. For example, the one or more electronic components can be located at the distal tip (a leading edge of the flexible elongate member and/or where the distal portion terminates) or proximally spaced from the distal end (by, e.g., 0.5 cm, 1 cm, 1.5 cm, 2 cm. 3 cm, 4 cm, 5 cm, and/or other suitable values both larger and smaller). Some embodiments of the intravascular device 102 include multiple, different electronic and/or sensing components (e.g., a pressure sensor and a flow sensor, or any other quantity or combination of sensors). In such embodiments, a first electronic component can be positioned at the distal tip of the flexible elongate member and the second electronic component can be spaced from the distal tip and/or from the first electronic component (by, e.g., 0.5 cm. 1 cm, 1.5 cm, 2 cm, 3 cm, 4 cm. 5 cm, and/or other suitable values both larger and smaller). In some embodiments, power, control signals, and electrical ground or signal return may be provided by the multi-filar conductor bundle 230, which includes multiple conductive filars. The conductive filars may, for example, be made of pure copper, or of a copper alloy such as BeCu or AgCu.

The intravascular device 102 includes a flexible elongate member 106. The sensing component 112 is disposed at the distal portion 107 of the flexible elongate member 106. The sensing component 112 can be mounted at the distal portion 107 within a housing 280 in some embodiments. A flexible tip coil 290 extends proximally from the housing 280 at the distal portion 107 of the flexible elongate member 106. A connection portion 114 located at a proximal end of the flexible elongate member 106 includes conductive portions 132, 134. In some embodiments, the conductive portions 132, 134 can be conductive ink that is printed and/or deposited around the connection portion 114 of the flexible elongate member 106. In some embodiments, the conductive portions 132, 134 are conductive, metallic rings that are positioned around the flexible elongate member. A locking section is formed by collar 118 and knob 120 are disposed at the proximal portion 109 of the flexible elongate member 106.

The intravascular device 102 in FIG. 1 includes a distal core wire 210 and a proximal core wire 220. The distal core 210 and the proximal core 220) are metallic components forming part of the body of the intravascular device 102. For example, the distal core 210 and the proximal core 220 are flexible metallic rods that provide structure for the flexible elongate member 106. The diameter of the distal core 210) and the proximal core 220) can vary along its length. A joint between the distal core 210) and proximal core 220 is surrounded and contained by a hypotube 215.

In some embodiments, the intravascular device 102 comprises a distal assembly and a proximal assembly that are electrically and mechanically joined together, which provides for electrical communication between the sensing component 112 and the conductive portions 132, 134. For example, flow data obtained by the sensing component 112 (in this example, sensing component 112 is a flow sensor) can be transmitted to the conductive portions 132, 134. Control signals (e.g., operating voltage, start/stop commands, etc.) from a processor system 306 in communication with the intravascular device 102 can be transmitted to the sensing component 112 via a connector 314 that is attached to the conductive portions 132, 134. The distal subassembly can include the distal core 210. The distal subassembly can also include the sensing component 112, a multi-filar conductor bundle 230), and/or one or more layers of insulative polymer/plastic 240 surrounding the conductive members 230) and the core 210. For example, the polymer/plastic layer(s) can insulate and protect the conductive members of the multi-filar cable or conductor bundle 230). The proximal subassembly can include the proximal core 220. The proximal subassembly can also include one or more layers of polymer layer(s) 250 (hereinafter polymer layer 250)) surrounding the proximal core 220) and/or conductive ribbons 260 embedded within the one or more insulative and/or protective polymer layer(s) 250. In some embodiments, the proximal subassembly and the distal subassembly can be separately manufactured. During the assembly process for the intravascular device 102, the proximal subassembly and the distal subassembly can be electrically and mechanically joined together. As used herein, flexible elongate member can refer to one or more components along the entire length of the intravascular device 102, one or more components of the proximal subassembly (e.g., including the proximal core 220, etc.), and/or one or more components the distal subassembly 210 (e.g., including the distal core 210, etc.). The joint between the proximal core 220 and distal core 210 is surrounded by the hypotube 215.

In various embodiments, the intravascular device 102 can include one, two, three, or more core wires extending along its length. For example, in one embodiment, a single core wire extends substantially along the entire length of the flexible elongate member 106. In such embodiments, a locking section 118 and a section 120 can be integrally formed at the proximal portion of the single core wire. The sensing component 112 can be secured at the distal portion of the single core wire. In other embodiments, such as the embodiment illustrated in FIG. 1, the locking section 118 and the section 120 can be integrally formed at the proximal portion of the proximal core 220. The sensing component 112 can be secured at the distal portion of the distal core 210. The intravascular device 102 includes one or more conductive members in a multi-filar conductor bundle 230) (e.g., a wire assembly) in communication with the sensing component 112. For example, the conductor bundle 230) can include one or more electrical wires that are directly in communication with the sensing component 112. In some instances, the conductive members 230) are electrically and mechanically coupled to the sensing component 112 by, e.g., soldering. In some instances, the conductor bundle 230 comprises two or three electrical wires (e.g., a bifilar cable or a trifilar cable). An individual electrical wire can include a bare metallic conductor, or a metallic conductor surrounded by one or more insulating layers. The multi-filar conductor bundle 230) can extend along a length of the distal core 210. For example, at least a portion of the conductive members 230) can be helically, or spirally, wrapped around an entire length of the distal core 210, or a portion of the length of the distal core 210).

The intravascular device 102 includes one or more conductive ribbons 260 at the proximal portion of the flexible elongate member 106. The conductive ribbons 260 are embedded within polymer layer(s) 250. The conductive ribbons 260 are directly in communication with the conductive portions 132 and/or 134. In some instances, the multi-filar conductor bundle 230 is electrically and mechanically coupled to the sensing component 112 by, e.g., soldering. In some instances, the conductive portions 132 and/or 134 comprise conductive ink (e.g., metallic nano-ink, such as silver or gold nano-ink) that is deposited or printed directed over the conductive ribbons 260).

As described herein, electrical communication between the conductive members 230) and the conductive ribbons 260) can be established at the connection portion 114 of the flexible elongate member 106. By establishing electrical communication between the conductor bundle 230 and the conductive ribbons 260, the conductive portions 132, 134 can be in electrically communication with the sensing component 112.

In some embodiments represented by FIG. 1, intravascular device 102 includes a locking section 118 and a section 120. To form locking section 118, a machining process is necessary to remove polymer layer 250 and conductive ribbons 260 in locking section 118 and to shape proximal core 220 in locking section 118 to the desired shape. As shown in FIG. 1, locking section 118 includes a reduced diameter while section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons.

In some embodiments, a connector 314 provides electrical connectivity between the conductive portions 132, 134 and a patient interface module or monitor 304. The patient interface module (PIM) 304 may in some cases connect to a console or processing system 306, which includes or is in communication with a display 308. In some embodiments, the patient interface module 304 includes signal processing circuitry, such as an analog-to-digital converter (ADC), analog and/or digital filters, signal conditioning circuitry, and any other suitable signal processing circuitry for processing the signals provided by the sensing component 112 for use by the processing system 306.

The system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some embodiments, device 102 may be controlled from a remote location such as the control room, such than an operator is not required to be in close proximity to the patient.

The intravascular device 102, PIM 304, and display 308 may be communicatively coupled directly or indirectly to the processing system 306. These elements may be communicatively coupled to the medical processing system 306 via a wired connection such as a standard copper multi-filar conductor bundle 230. The processing system 306 may be communicatively coupled to one or more data networks, e.g., a TCP/IP-based local area network (LAN). In other embodiments, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed on the display 308. The console or processing system 306 can include a processor and a memory. The processing system 306 may be operable to facilitate the features of the intravascular sensing system 100 described herein. For example, the processor can execute computer readable instructions stored on the non-transitory tangible computer readable medium.

The PIM 304 facilitates communication of signals between the processing system 306 and the intravascular device 102. In some embodiments, the PIM 304 performs preliminary processing of data prior to relaying the data to the processing system 306. In examples of such embodiments, the PIM 304 performs amplification, filtering, and/or aggregating of the data. In an embodiment, the PIM 304 also supplies high- and low-voltage DC power to support operation of the intravascular device 102 via the multi-filar conductor bundle 230).

The multi-filar cable or transmission line bundle 230 can include a plurality of conductors, including one, two, three, four, five, six, seven, or more conductors. The multi-filar conductor bundle 230 can be positioned along the exterior of the distal core 210. The multi-filar conductor bundle 230 and the distal core 210 can be overcoated with an insulative and/or protective polymer 240. In the example shown in FIG. 1, the multi-filar conductor bundle 230) includes two straight portions 232 and 236, where the multi-filar conductor bundle 230) extends linearly and parallel to a longitudinal axis of the flexible elongate member 106 on the exterior of the distal core 210, and a helical or spiral portion 234, where the multi-filar conductor bundle 230 is wrapped around the exterior of the distal core 210. In some embodiments, the multi-filar conductor bundle 230 only includes a straight portion or only includes a helical or spiral portion. In general, the multi-filar conductor bundle 230 can extend in a linear, wrapped, non-linear, or non-wrapped manner, or any combination thereof. Communication, if any, along the multi-filar conductor bundle 230 may be through numerous methods or protocols, including serial, parallel, and otherwise, wherein one or more filars of the bundle 230 carry signals. One or more filars of the multi-filar conductor bundle 230 may also carry direct current (DC) power, alternating current (AC) power, or serve as an electrical ground connection.

The display or monitor 308 may be a display device such as a computer monitor, a touch-screen display, a television screen, or any other suitable type of display. The monitor 308 may be used to display selectable prompts, instructions, and visualizations of imaging data to a user. In some embodiments, the monitor 308 may be used to provide a procedure-specific workflow to a user to complete an intraluminal imaging procedure.

Before continuing, it should be noted that the examples described above are provided for purposes of illustration, and are not intended to be limiting. Other devices and/or device configurations may be utilized to carry out the operations described herein.

As described above, the sensing component 112 may include a transducer, such as an ultrasound transducer, configured to transmit acoustic (e.g., ultrasound) energy. In some embodiments, the sensing component 112 may further include an acoustic matching layer, which may aid in the propagation of the ultrasound energy transmitted from the sensing component. For instance, the acoustic matching layer may minimize acoustic impedance mismatch between the ultrasound transducer and a sensed medium, such as a fluid and/or a lumen that the intravascular device 102 is positioned within. In this regard, properties of the acoustic matching layer, including dimensions (e.g., a thickness) of the acoustic matching layer, may impact a performance of the sensing component 112 (e.g., an accuracy, a precision, and/or resolution of data obtained by the sensing component 112). As such, variations in acoustic matching layer dimensions across different devices (e.g., devices 102) may result in unreliable and/or inconsistent performance of the sensing component within the devices. Accordingly, the present disclosure relates to mechanisms for controlling a thickness of an acoustic matching layer applied to the sensing component 112.

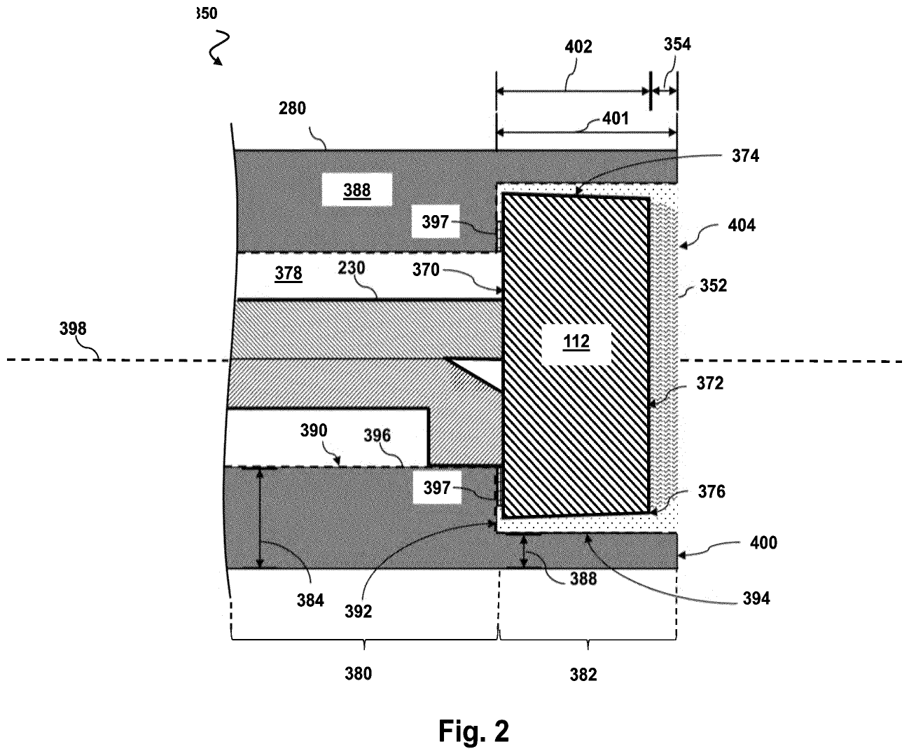

Turning now to FIG. 2, a diagrammatic cross-sectional view of an example sensor assembly 350, which may be included in the intravascular device 102 of FIG. 1, is shown. More specifically, FIG. 2 illustrates a sensor assembly 350 that includes a sensing component 112, a housing 280, and an acoustic matching layer 352 that has a thickness 354, which may be a predetermined (e.g., set) thickness. As indicated by the positions of the sensing component 112 and the housing 280 illustrated in FIG. 1, the sensor assembly 350 may be included in a distal portion of the intravascular device 102 such that the surface 372 of the sensing component 112 faces distally.

As illustrated in FIG. 2, the sensing component 112 is positioned within the housing 280 and includes a proximal surface 370, an opposite, distal surface 372, and a side surface 374. In some embodiments, one or more of the proximal surface 370), the distal surface 372, or the side surface 374 may be coated in an insulating layer 376. The insulating layer 376 may be formed from parylene, which may be deposited on the one or more surfaces, for example. The insulating layer 376 may additionally or alternatively be formed from any other suitable insulating material. In some embodiments, the insulating layer 376 may prevent a short (e.g., an electrical failure), which may otherwise be caused by contact between a conductive portion of the sensing component 112 and the housing 280, which may be formed with a metal. As used herein, references to the distal surface 372 encompass the insulating layer 376 in embodiments where a distal end of the sensing component 112 is covered by the insulating layer 376, references to the proximal surface 370) encompass the insulating layer in embodiments where a proximal end of the sensing component 112 is covered by the insulating layer 376, and references to the side surface 374 encompass the insulating layer in embodiments where the side of the sensing component 112 is covered by the insulating layer 376 unless indicated otherwise.

In some embodiments, the sensing component 112 may include a transducer element, such as an ultrasound transducer element on the distal surface 372 such that the transducer element faces distally and may be used by the sensing component 112 to obtain sensor data corresponding to a structure distal of the sensing component 112. The sensing component 112 may additionally or alternatively include a transducer element on the proximal surface 370 such that the transducer faces proximally and may be used to obtain sensor data corresponding to a structure proximal of the sensing component. A transducer element may additionally or alternatively be positioned on a side surface 374 (e.g., on a perimeter or circumference) of the sensing component 112 in some embodiments.

As further illustrated, the sensing component 112 is coupled to the multi-filar conductor bundle 230), and at least a portion (e.g., a distal portion) of the multi-filar conductor bundle 230) are extends through the housing 280. In some embodiments, the multi-filar conductor bundle 230) and the sensing component 112 may be physically (e.g., mechanically) coupled. Further, one or more filars (e.g., conductive members) of the multi-filar conductor bundle 230 may electrically couple to (e.g., be in electrical communication) with the sensing component 112. In particular, one or more filars of the multi-filar conductor bundle 230 may couple to an element, such as a transducer (e.g., an ultrasound transducer), of the sensing component 112 and may provide power, control signals, an electrical ground or signal return, and/or the like to the element. As described above, such an element may be positioned on the distal surface 372 of the sensor. In that regard, in some embodiments, one or more filars of the multi-filar conductor bundle 230 may extend through a cutout or hole in the sensing component 112 (e.g., in at least the proximal surface 370)) to establish electrical communication with an element on the distal surface 372 of the sensor. Filars may additionally or alternatively wrap around the side surface 374 to establish electrical communication with the element on the distal surface 372. Moreover, in some embodiments, filars of the multi-filar conductor bundle 230) may terminate at and/or electrically couple to the proximal surface 370) (e.g., to an element on the proximal surface 370) of the sensing component 112. Further, in some embodiments, a subset of the filars of the multi-filar conductor bundle 230) may extend to the distal surface 372 and/or electrically couple to an element at the distal surface 372, while a different subset of the filars may electrically couple to an element at the proximal surface 370, for example.

In some embodiments, the multi-filar conductor bundle 230) may be coated in the insulating layer 376. In some embodiments, for example, the multi-filar conductor bundle 230) and the sensing component 112 may be coupled together in a sub-assembly before being positioned in the housing 280. In such embodiments, the insulating layer 376 may be applied (e.g., coated and/or deposited) onto the entire sub-assembly, resulting in an insulating layer 376 on both the sensing component 112 and the multi-filar conductor bundle 230).

The housing 280 may include a hollow interior 378, as well as a proximal portion 380 and a distal portion 382. The hollow interior 378 may be defined by a continuous surface (e.g., an integrally formed surface), which includes an inner distal surface 394, an inner proximal surface 390, and a planar surface 392. In some embodiments, housing 280) and the continuous surface may be formed via an additive process such that the housing 280 is a unitary component and features of the housing 280 may be formed with micron-level precision, as described in greater detail below.

As shown, a shape of the hollow interior 378 within the proximal portion 380 may vary from the shape of the hollow interior 378 within the distal portion 382. In particular, as illustrated, a thickness 384 (e.g., an inner diameter) of a wall 386 of the housing 280 at the proximal portion 380 may be different (e.g., thicker) than a thickness 388 of the wall 386 at the distal portion 382. In this regard, the hollow interior 378 includes a counterbore 396 (outlined with dashed lines), which includes the planar surface 392 and a thru-hole (e.g., the portion of the hollow interior 378 distal of the planar surface 392). The counterbore 396 is arranged to receive the sensing component 112 within the distal portion 382 (e.g., in the portion of the hollow interior 378 distal of the planar surface 392) and to receive the multi-filar conductor bundle 230 within the proximal portion 380) (e.g., within the thru-hole).

As further illustrated, the sensing component 112 is positioned on the planar surface 392. In some embodiments, the sensing component 112 may be positioned directly on the planar surface 392. In other embodiments, an adhesive 397 may be positioned between the sensing component 112 and the planar surface 392. The adhesive 397 may secure the sensing component 112 to the housing 280, for example. In any case, the proximal surface 370) of the sensing component 112 is positioned on (e.g., directly or indirectly) the planar surface 392. Moreover, the proximal surface 370) and the planar surface 392 are arranged such that the proximal surface 370 is parallel with the planar surface 392 across the entirety of the planar surface 392. As such, the sensing component 112 may be aligned such that the proximal surface 370) and/or the distal surface 372 are perpendicular with a longitudinal axis 398 of the housing 280. Thus, the arrangement of the proximal surface 370 and the planar surface 392 may align of a transducer (e.g., an ultrasound transducer) on the distal surface 372 perpendicular to the longitudinal axis 398 and may prevent misalignment of the transducer.

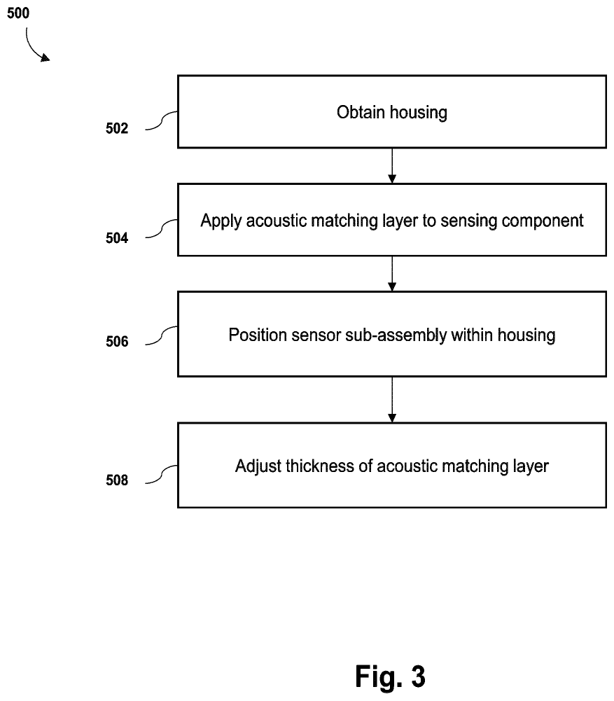

In some embodiments, the acoustic matching layer 352 may be positioned on (e.g., over) the distal surface 372 of the sensing component 112. In particular, the acoustic matching layer 352 may be disposed directly on the sensing component 112, or the acoustic matching layer 352 may be disposed on the insulating layer 376 coating the sensing component 112. Further, the acoustic matching layer 352 may be disposed on a transducer element (e.g., an ultrasound transducer element) positioned on the sensing component (e.g., the distal surface 372) and/or at least a portion of a conductive filar of the multi-filar conductor bundle 230) that is in communication with the transducer element, such as a filar extending through a hole or along a side of the sensing component 112. To that end, the acoustic matching layer 352 may contact and/or at least partially surround the portion of the conductive filar and/or the transducer element. Moreover, the acoustic matching layer 352 may provide acoustic matching to the sensing component 112 (e.g., to an ultrasound transducer of the sensing component 112). For instance, the acoustic matching layer 352 may minimize acoustic impedance mismatch between the ultrasound transducer and a sensed medium, such as a fluid and/or a lumen that the intravascular device 102 is positioned within. In that regard, the acoustic matching layer 352 may be formed from any suitable material, such as a polymer or an adhesive, to provide acoustic matching with the sensing component 112. Moreover, the acoustic matching layer 352 may include and/or be formed from the same material as the adhesive 397 or a different material than the adhesive 397. The acoustic matching layer 352 and/or an additional adhesive may further be positioned between the side surface 374 and the inner distal surface 394. As illustrated, the portion of the acoustic matching layer 352 positioned on the distal surface 372 may include a first material (illustrated by a first fill pattern), while the portion of the acoustic matching layer positioned on the side surface 374 and/or the proximal surface 370 may be include a different, second material (illustrated by a second fill pattern). However, embodiments are not limited thereto. For instance, the portion of the acoustic matching layer 352 positioned on the distal surface 372 may include and/or be formed from the same material as the portion of the acoustic matching layer positioned on the side surface 374 and/or the proximal surface 370. Further, as described in greater detail with respect to FIGS. 3-7, the acoustic matching layer 352 may be applied to the sensing component 112 before or after the sensing component 112 is positioned within the housing 280 during assembly of the sensor assembly 350. In this regard, the portion of the acoustic matching layer 352 positioned on the distal surface 372 and the portion of the acoustic matching layer positioned on the side surface 374 and/or the proximal surface 370) may be included in the sensor assembly 350 in the same or different steps. Further, in addition to the one or more materials the acoustic matching layer 352 is formed from, the acoustic matching layer 352 may provide acoustic matching with the sensing component 112 via one or more dimensions of the acoustic matching layer 352. In particular, the thickness 354 of the acoustic matching layer 352 between the distal surface 372 and the distal end 400 of the housing 280 may be controlled (e.g., set and/or tuned) to affect particular acoustic matching characteristics (e.g., impedance matching) with respect to the sensing component 112, as described below. In some embodiments, for example, the thickness 354 may be a predetermined thickness and/or acoustic matching characteristics of the acoustic matching layer 352 may be predetermined. More specifically, the thickness 354 may be set (e.g., predetermined) based on dimensioning of the housing 280 and the sensing component 112, for example.

In some embodiments, the sensor assembly 350) may include an atraumatic tip, such as the distal tip 108 illustrated in FIG. 1. In some embodiments, the distal tip 108 may include the same material as the acoustic matching layer 352. In some embodiments, the distal tip may include a different material than the acoustic matching layer 352. Additionally or alternatively the distal tip 108 may be formed from one or more layers of materials. The layers may include different materials and/or different configurations (e.g., shape and/or profile, thickness, and/or the like). Further, the distal tip 108 may be arranged to cover the distal surface 372 of the sensing component 112. In some embodiments, the distal tip 108 may also cover a distal end 410 of the housing 280. Moreover, while the distal tip 108 is illustrated as having a domed shape, embodiments are not limited thereto. In this regard, the distal tip 108 may include a flattened profile or any suitable shape.

In some embodiments, the housing 280) may be arranged such that the planar surface 392 is spaced from the distal end 400 of the housing 280 along the longitudinal axis 398 by a distance 401, which exceeds a distance 402 along the longitudinal axis between the planar surface 392 and the distal surface 372 of the sensing component 112. In this regard, the entire sensing component 112 may be positioned within (e.g., surrounded by the continuous surface of) the housing 280. Moreover, the distal end 400 of the housing 280 may be spaced from the distal surface 372 of the sensing component 112 along the longitudinal axis 398. As illustrated, for example, the distal surface 372 may be spaced from the sensing component 112 by a distance indicated by the thickness 354. As described herein, the acoustic matching layer 352 may be positioned on (e.g., over) the distal surface 372 of the sensing component 112. In this way, the thickness 354 (e.g., along the longitudinal axis 398) of the acoustic matching layer 352 may be defined by (e.g., set and/or controlled by) the distance between the distal surface 372 and the distal end 400. In particular, in cases where a distal end 404 of the acoustic matching layer is coplanar (e.g., flush) with the distal end 400) of the housing 280, as illustrated, the total thickness (e.g., thickness 354) of the acoustic matching layer 352 may be determined by the distance between the distal surface 372 and the distal end 400. While the acoustic matching layer 352 is illustrated as being flush (e.g., coplanar) with the distal end 400, embodiments are not limited thereto. In this regard, even if a first portion of the acoustic matching layer 352 extends past the distal end 400 with respect to the longitudinal axis 398, the thickness of a second portion of the acoustic matching layer 352 that is proximal of the distal end 400 is defined by the illustrated thickness 354.

Thus, at least a portion of the thickness (e.g., the thickness 354) of the acoustic matching layer 352 may be controlled by the configuration of different components of the sensor assembly 350. For instance, the thickness 354 may vary based on the distance 401 between the distal end 400 and the planar surface 392, which may be determined based on a size of the distal portion 382 of the housing 280 (e.g., a length of the inner distal surface 394). Moreover, the thickness 354 may vary based on the distance 402 between the distal surface 372 of the sensing component 112 and the planar surface 392. The distance 402 may be affected by the length of the inner distal surface 394, the dimensions of the sensing component 112, and/or the configuration of components, such as the adhesive 397, between the sensing component 112 and the planar surface 392, for example. Further, in some embodiments, the housing 280 may be formed via a manufacturing process that utilizes micron-level precision, such as an additive formation process described in greater detail below. As such, the dimensions of the housing 280 and, as a result, the thickness of the 354 may be relatively precisely (e.g., at least a micron-level) controlled. In this way, the thickness 354 of the acoustic matching layer 352 may be controlled to provide certain performance characteristics at the sensing component 112, and these performance characteristics may be relatively consistent across different devices (e.g., intravascular devices 102) formed according to the same arrangement of components within the sensor assembly 350).

With reference now to FIG. 3, a flow diagram of a method 500 of assembling a sensor assembly, such as sensor assembly 350 of FIG. 2 is shown, according to aspects of the preset disclosure. In some embodiments, the sensor assembly assembled according to the method 500 may be positioned within an intraluminal sensing device, such as intravascular device 102 of FIG. 1. As illustrated, the method 500) includes a number of enumerated steps, but embodiments of the method 500 may include additional steps before, after, or in between the enumerated steps. In some embodiments, one or more of the enumerated steps may be omitted, performed in a different order, or performed concurrently.

At step 502, the method 500 may include obtaining a housing, such as housing 280. In particular, a housing terminating in a distal end (e.g., distal end 400) and including a hollow interior (e.g., hollow interior 378) with a planar surface (e.g., planar surface 392) may be obtained, as described with respect to FIG. 2. In this regard, the hollow interior may be defined by a counterbore (e.g., counterbore 396) that includes the planar surface and a thru-hole (e.g., the portion of the hollow interior 378 proximal of the planar surface 392). Moreover, a length of an inner distal surface (e.g., inner distal surface 394) of the housing with respect to a longitudinal axis of the housing (e.g., longitudinal axis 398) may exceed a length between a proximal surface (e.g., proximal surface 370)) and a distal surface (e.g., distal surface 372) of a sensing component (e.g., sensing component 112).

In some embodiments, the obtained housing may be formed via an additive manufacturing process, as described above. In particular, the housing may be constructed from a manufacturing method, such as three-dimensional (3D) printing, photolithography, electrodeposition, and/or other suitable processes (e.g., micro-electromechanical system (MEMs) and/or semiconductor manufacturing processes), that involves forming the housing from a plurality of layers. For instance, the plurality of layers may be formed atop one another according to any combination of the above manufacturing techniques such that the layers define a continuous (e.g., integral) surface of the housing. In this way, the housing may be formed as a unitary component. Further, the continuous surface of the housing may be formed via the additive process to include an inner proximal surface, an inner distal surface, and a planar surface (e.g., the inner proximal surface 390), the inner distal surface 394, and the planar surface 392). Further, in some embodiments, individual layers of the plurality of layers may be formed with different materials, thicknesses, height, lengths, shapes, and/or like as one another. In this way, characteristics of the housing may be tuned via selection of the layers used to form the housing. For instance, dimensions of the inner proximal surface, the inner distal surface, and/or the planar surface of the housing may be tuned based on the arrangement of the layers. Moreover, in some embodiments, these dimensions may be tuned at the precision of at least a micron. While a housing formed via an additive manufacturing process has been described herein, embodiments are not limited thereto. In this regard, in some embodiments, the obtained housing may be formed via a subtractive process (e.g., involving drilling, cutting, and/or the like) or any suitable combination of manufacturing processes.

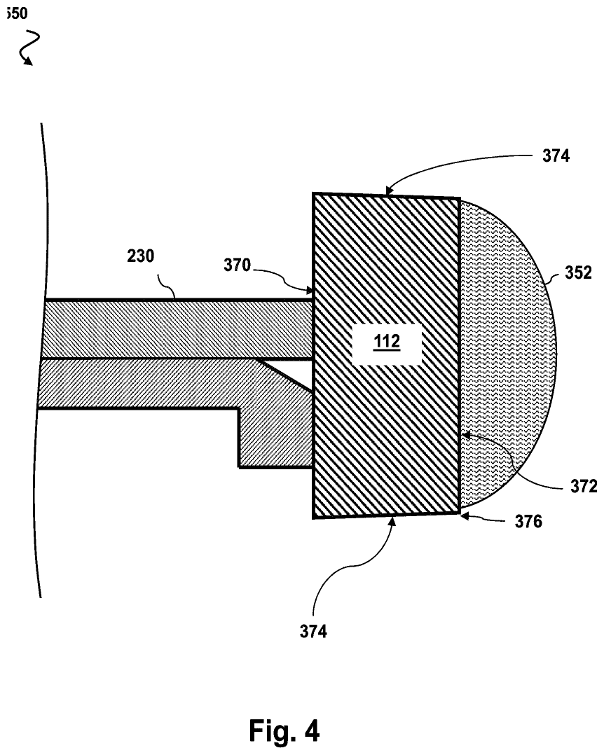

At step 504, the method 500 may involve applying an acoustic matching layer (e.g., acoustic matching layer 352) to a sensing component (e.g., sensing component 112). Applying the acoustic matching layer may involve depositing the acoustic matching layer, coating the acoustic matching layer, patterning the acoustic matching layer, and/or the like upon one or more surfaces of the sensing component. Additionally or alternatively, the sensing component may be dipped into an acoustic matching material (e.g., an adhesive) to form the acoustic matching layer on the sensing component. For instance, the acoustic matching layer may be applied to a distal surface (e.g., distal surface 372) of the sensing component. In some embodiments, the acoustic matching layer may be applied the side surface (e.g., side surface 374) and/or the proximal surface (e.g., proximal surface 370)) of the sensing component. Moreover, according to the method 500, the acoustic matching layer may be applied to the one or more surfaces of the sensing component before the sensing component is positioned within the obtained housing. In this regard, the acoustic matching layer or a portion thereof may be applied to a sensor sub-assembly, which may include the sensing component coupled to a multi-filar conductor bundle (e.g., multi-filar conductor bundle 230), as illustrated in FIG. 4.

Turning now to FIG. 4, a diagrammatic cross-sectional view of an example sensor sub-assembly 550, which may be included in the sensor assembly 350 of FIG. 2, is shown. The sensor sub-assembly 550 includes the sensing component 112 with an acoustic matching layer 352 coupled to the multi-filar conductor bundle 230). In some embodiments, the acoustic matching layer 352 may be positioned on (e.g., disposed atop) at least a portion of one or more conductive filars of the multi-filar conductor bundle 230. For instance, the acoustic matching layer 352 may be positioned on (e.g., in contact with) a transducer element of the sensing component 112, as well as at least a portion of a filar in communication with (e.g., communicatively coupled to) the transducer element. Further, in some embodiments, the sensor sub-assembly 550 may be obtained in accordance with step 504 of the method 500) of FIG. 3.

In the illustrated embodiment, the acoustic matching layer 352 is positioned on the distal surface 372 of the sensing component 112. In particular, the acoustic matching layer 352 is illustrated as covering the entire distal surface 372. Moreover, the acoustic matching layer 352 is shown as having a relatively regular shape (e.g., a domed profile). However, embodiments are not limited thereto. In this regard, the acoustic matching layer 352 may be applied to any combination of the sides (e.g., 370, 374, or 376) of the sensing component 112. In addition, the acoustic matching layer 352 may be applied to this combination of sides in any suitable shape or configuration. For instance, the acoustic matching layer 352 may be applied such that the acoustic matching layer has a relatively flat (e.g., planar profile), a relatively irregular (e.g., undefined and or amorphous) shape, and/or any other shape. As an illustrative example, applying the acoustic matching layer 352 to the sensing component 112 of the sensor sub-assembly 550) (e.g., at step 504 of FIG. 3) by dipping the sensing component 112 into acoustic matching material may produce an acoustic matching layer 352 with an amorphous shape. As a further example, applying the acoustic matching layer 352 to the sensing component 112 by coating or depositing the acoustic matching layer 352 on the sensing component may produce an acoustic matching layer 352 with a more defined and/or controllable shape.

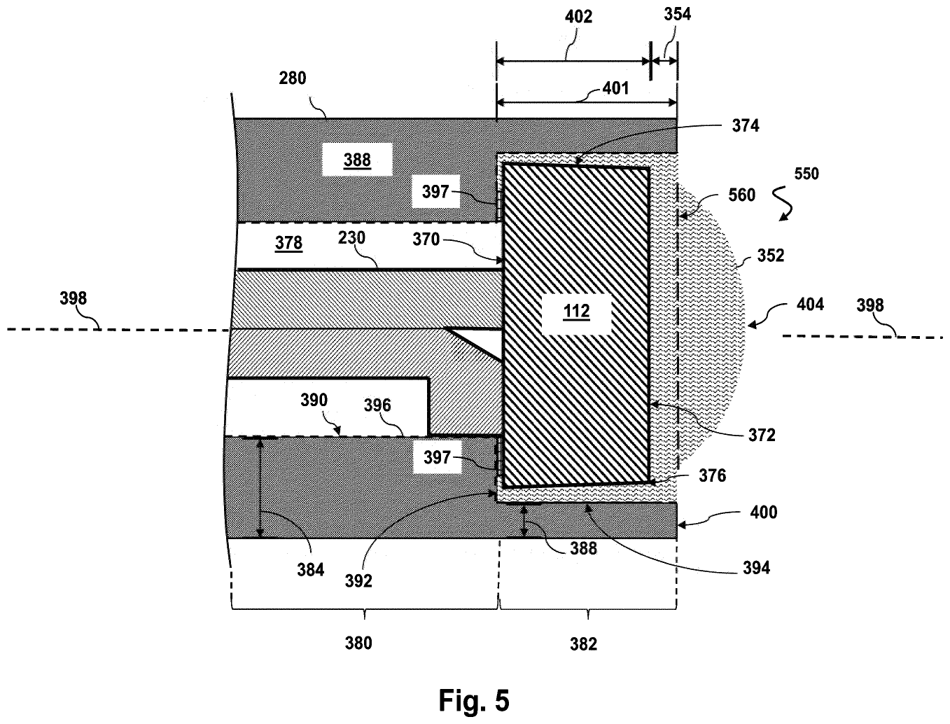

With reference now to FIG. 3, at step 506 the method 500 may involve positioning a sensor sub-assembly within the housing. In particular, the step 506 may involve positioning a sensor sub-assembly, such as the sensor sub-assembly 550 of FIG. 4, that includes the acoustic matching layer applied to the sensing component of the sensor sub-assembly within the housing. Moreover, positioning the sensor sub-assembly within the housing may involve sliding the distal end (e.g., distal end 400) of the housing over the proximal end of the sensor sub-assembly (e.g., over the multi-filar conductor bundle 230)) such that the sensor sub-assembly is positioned within the hollow interior of the housing. More specifically, the sensor sub-assembly may be positioned such that the multi-filar conductor bundle of the sensor sub-assembly is positioned within a proximal portion of the housing and the sensing component of the sensor sub-assembly is positioned within a distal portion of the housing upon the planar surface of the housing. The sensor sub-assembly may further be secured within the housing, once positioned within the housing, by an adhesive (e.g., adhesive 397) positioned between the housing and the sensing component. The adhesive may be positioned within the housing before the sensor sub-assembly is positioned within the housing in some embodiments, for example. Additionally or alternatively, the adhesive may be positioned within the housing after the sensor sub-assembly is positioned within the housing. An example of the sub-assembly positioned within the housing in accordance with step 506 is illustrated in FIG. 5.

FIG. 5 is a diagrammatic cross-sectional view of an example sensor sub-assembly 550 positioned within the housing 280, in accordance with step 506 of the method 500 of FIG. 3. The sensor sub-assembly 550) includes the sensing component 112 with an acoustic matching layer 352 coupled to the multi-filar conductor bundle 230. In this regard, the sensor sub-assembly 550 may be obtained in accordance with step 504 of the method 500 of FIG. 5, in some embodiments.

As illustrated in FIG. 5 and described above, a portion of the acoustic matching layer 352 positioned between the side surface 374 of the sensing component 112 and housing 280 (e.g., the inner distal surface 394) may be formed from the same material as a portion of the acoustic matching layer 352 positioned over the distal surface 372. As illustrated in FIG. 1, these portions of the acoustic matching layer 352 may alternatively be formed from different materials. In any case, the portion of the layer 352 positioned between the side surface 374 of the sensing component 112 and housing 280 may secure the sensor sub-assembly 550 to the housing 280. For instance, the acoustic matching layer 352 may be cured such that the sensing component 112 is secured to the housing 280. Accordingly, positioning the sensor sub-assembly 550 within the housing 280 may involve positioning a portion of the acoustic matching layer 352 between the sensing component 112 and the housing 280.

In some embodiments, positioning the sensor sub-assembly 550 within the housing 280 may distribute the acoustic matching layer 352 applied to the sensor sub-assembly 550. For instance, the acoustic matching layer 352 may cover one or more surfaces of the sensing component 112 such that, when the sensing component 112 is positioned within the housing 280, the acoustic matching layer 352 forms to (e.g., flows into) the space between the housing 280 and the sensing component 112. In other embodiments, adhesive and/or an acoustic matching material, which may be the same as or different from the adhesive 397, may be applied within the housing 280. This adhesive and/or acoustic matching material may be applied within the housing 280 before or after the sensing component 112 is positioned within the housing, or both. For instance, in some embodiments acoustic matching material may be applied within the housing 280 such that, when the sensing component 112 is positioned within the housing 280, the acoustic matching material forms to (e.g., flows into) the space between the housing 280 and the sensing component 112. When cured, this material may form the acoustic matching layer together with acoustic matching material applied to the sensor sub-assembly 550). In some embodiments, the sensor sub-assembly 550 with an acoustic matching layer 352 applied on the distal surface 372, as illustrated in FIG. 4, may be positioned within the housing 280. Subsequently, space between the housing 280 and the sensing component 112 may be filled by additional acoustic matching material, which may form a first portion of the acoustic matching layer 352, and the acoustic matching material previously formed on the distal surface 372 may form a second portion of the acoustic matching layer 352.

Turning back now to FIG. 3, at step 508, the method 500 may involve adjusting a thickness of the acoustic matching layer. In some embodiments, for example, a desired configuration of the acoustic matching layer includes the distal end of the acoustic matching layer being coplanar (e.g., flush) with the distal end of the housing such that the thickness of the acoustic matching later extending from the distal surface of the sensing component is defined by the sensing component and the housing (e.g., thickness 354), as illustrated in FIG. 2. However, as illustrated in FIG. 5, in some cases the distal end of the acoustic matching layer may be distal of the distal end of the housing, causing the thickness of the acoustic matching layer to exceed the thickness 354. Accordingly, manufacture of the sensor assembly 350 (e.g., in accordance with method 500 of FIG. 3) may involve removing or adjusting a thickness of the acoustic matching layer 352 in excess of the thickness 354. In that regard, the acoustic matching layer may be cut, trimmed, or sanded, and/or a portion of the acoustic matching layer may be removed via a lapping film, via application of a chemical (e.g., a chemical abrasive), or any other suitable method. In particular, the portion of the acoustic matching layer exceeding distal end 400 of the housing 280, as indicated by the dashed guide line 560 of FIG. 5, may be removed. To that end, the distal end 400 may serve as a visual and/or a physical (e.g., mechanical) guide to precisely trim and/or configure the acoustic matching layer 352 such that the distal end 404 of the acoustic matching layer 352 is flush (e.g., coplanar) with the distal end 400 of the housing 280) and the thickness of the acoustic matching layer 352 extending from the distal surface 372 is defined by the thickness 354. In this way, the thickness of the acoustic matching layer 352 may be trimmed or reduced to a predetermined (e.g., known) thickness (e.g., thickness 354). Moreover, by adjusting the thickness of the acoustic matching layer 352, the assembly illustrated in FIG. 5 may be modified to be substantially similar to the sensor assembly 350 described with respect to FIG. 2.

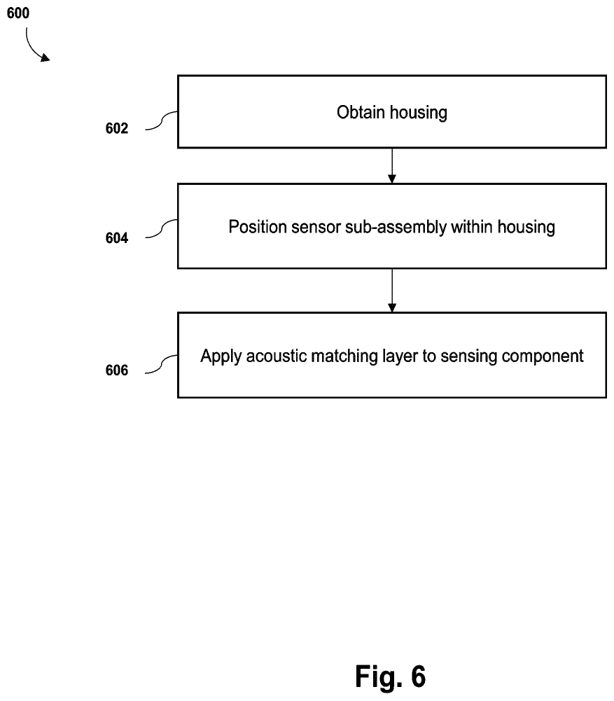

With reference now to FIG. 6, a flow diagram of a method 600 of assembling a sensor assembly, such as sensor assembly 350 of FIG. 2 is shown, according to aspects of the preset disclosure. In some embodiments, the sensor assembly assembled according to the method 600 may be positioned within an intraluminal sensing device, such as intravascular device 102 of FIG. 1. As illustrated, the method 600 includes a number of enumerated steps, but embodiments of the method 600 may include additional steps before, after, or in between the enumerated steps. In some embodiments, one or more of the enumerated steps may be omitted, performed in a different order, or performed concurrently.

At step 602, the method 600 may involve obtaining a housing, such as the housing 280. The step 602 of the method 600 may be substantially similar to the step 502 of the method 500 illustrated in FIG. 5. Thus, for the sake of brevity, details of obtaining a housing described above with reference to FIG. 3, will not be repeated.

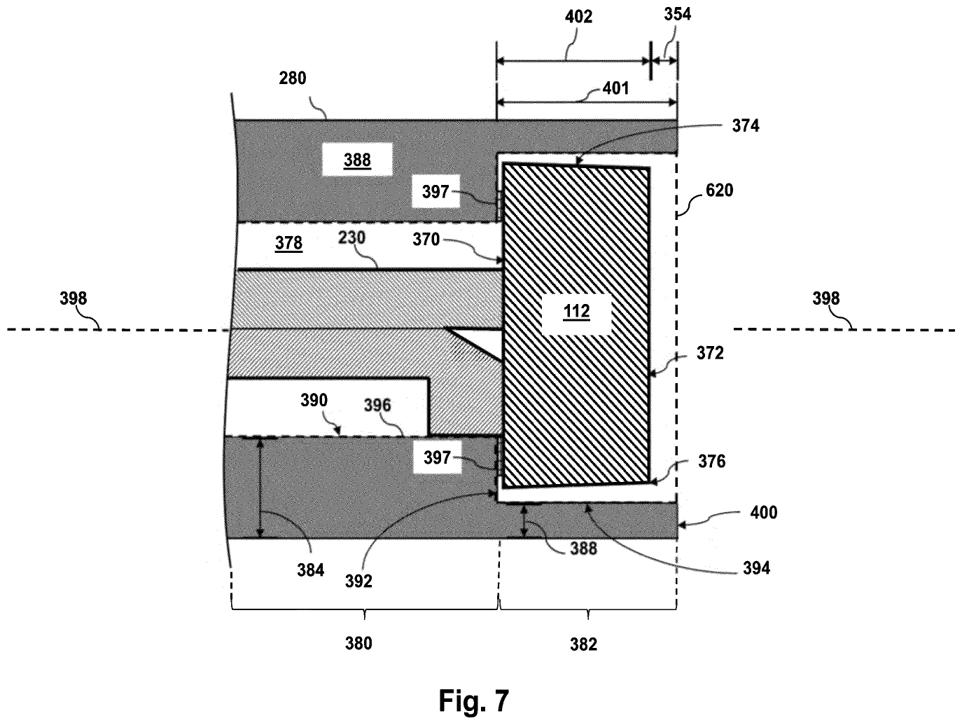

At step 604, the method 600 may involve positioning a sensor sub-assembly within the housing. In particular, a sensor sub-assembly that includes a sensing component (e.g., sensing component 112) coupled to a multi-filar bundle (e.g., multi-filar conductor bundle 230) and lacks an acoustic matching layer (e.g., acoustic matching layer 352) may be positioned within the housing. To that end, in contrast with the method 500 of FIG. 5, the method 600 may involve positioning the sensor sub-assembly within the housing before an acoustic matching layer is applied to the sensor sub-assembly. An example of a sub-assembly positioned within the housing in accordance with the method 600 is illustrated in FIG. 7.

FIG. 7 is a diagrammatic cross-sectional view of an example sensor sub-assembly positioned within the housing 280, in accordance with step 604 of the method 600 of FIG. 6. As illustrated, the sensor sub-assembly includes the sensing component 112 coupled to the multi-filar conductor bundle 230 and lacks an acoustic matching layer (e.g., acoustic matching layer 352). In some embodiments, the housing 280 may include acoustic matching material that may form a portion of an acoustic matching layer 352. To that end, positioning the sensor sub-assembly within the housing at step 604 of the method 600 may involve positioning the sensing component such that the acoustic matching material within the housing is applied to one or more sides of the sensing component 112. Moreover, positioning the sensing component 112 within the housing 280 may involve securing the sensing component 112 to the housing 280 via the adhesive 397, as described herein.

Turning back now to FIG. 6, at step 606, the method 600 may involve applying an acoustic matching layer to the sensing component. With reference to FIG. 7, applying the acoustic matching layer may involve filling at least a portion of the space 620 defined between the housing 280 and the sensing component 112 with the acoustic matching layer. In some embodiments, for example, the acoustic matching layer may be applied, using one or more materials, to the sensing component 112 to such that the acoustic matching layer has a thickness 354 between the distal surface 372 and the distal end 400. In particular, the acoustic matching layer may be applied to be flush (e.g., coplanar) with the distal end 400). To that end, the process of filling the space 620 may be visually and/or physically guided by the distal end 400 of the housing 280. In this way, the thickness of the acoustic matching layer 352 may be set to a preset (e.g., known) thickness (e.g., thickness 354). In some embodiments, application of the acoustic matching layer 352 (e.g., filling of the space 620)) may involve applying the acoustic matching layer 352 such that the acoustic matching layer 352 is positioned on (e.g., covers and/or contacts) at least a portion of a transducer element (e.g., ultrasound transducer element) of the sensing component 112 and/or at least a portion of a filar of the multi-filar conductor bundle 230), such as a filar extending through a hole in the sensing component 112. In particular, the sensing component 112 and the at least portion of the filar may be positioned on the distal surface 372, which may be covered with the acoustic matching layer 352. Further, in some embodiments, excess acoustic matching material may be removed or adjusted, as described above with reference to step 508 of the method 500 of FIG. 3.

Embodiments described herein are intended to be exemplary and not limiting. In this regard, one or more of the illustrated components in a sensor assembly may be omitted, additional components may be added, and two components illustrated as separate may represent a single component. Further, while the housing 280 is illustrated as having a relatively constant outer profile, embodiments are not limited thereto. To that end, the outer profile may have a different height (e.g., perpendicular to the longitudinal axis 398) at the distal portion 382 than the proximal portion 380 in some embodiments. Further, in some embodiments, the adhesive 397 may be positioned in the housing 280 such that the adhesive 397 contacts the side surface 374 of the sensing component. In this regard, the adhesive 397 may be positioned at any suitable position or combination of positions within the housing 280. Moreover, the hollow interior 378 may be filled or partially filled with a material within the proximal portion 380. For instance, the multi-filar conductor bundle 230 may be at least partially surrounded by air, an adhesive, an acoustic backing material, and/or the like within the proximal portion 380.

Moreover, while the sensing component 112 and the housing 280 are illustrated as having a particular configuration (e.g., shape, structural arrangement, and/or the like), embodiments are not limited thereto. In this regard, while the sensing component 112 is illustrated and described as having a trapezoidal profile in the side views illustrated in FIGS. 2, 4, 5, and 7, the sensing component 112 may have any suitable shape or dimensions. For instance, the sensing component 112 may include one or more planar, spherical, or cylindrical portions. Moreover, the sensing component 112 may a rectangular profile with respect to the side views of FIGS. 2, 4, 5, and 7, or the sensing component 112 may be arranged such that the height of the sensing component 112 increases moving proximally to distally. Further, while the housing 280 is illustrated and described as being cylindrical, the housing 280 may have any suitable shape and/or dimensions. For instance, the housing 280) may include one or more planar portions. Accordingly, references to circular, cylindrical, annular configurations and/or dimensions are intended to be exemplary and not limiting.

A person of ordinary skill in the art will recognize that the present disclosure advantageously provides a sensor assembly that controls a thickness of an acoustic matching layer positioned on a sensing component. In particular, the thickness of the acoustic matching layer may be defined by a relationship between dimensions of the sensing component and dimensions of a housing that the sensing component is positioned within. Because the performance of the sensing component (e.g., the performance of the ultrasound transducer) may depend on the thickness of the acoustic matching layer, defining the thickness of the acoustic matching layer on components (e.g., the sensing component and the housing) with a fixed relationship may reduce or prevent inconsistencies in this performance. The logical operations making up the embodiments of the technology described herein are referred to variously as operations, steps, objects, elements, components, regions, etc. Furthermore, it should be understood that these may occur in any order, unless explicitly claimed otherwise or a specific order is inherently necessitated by the claim language.

It should further be understood that the described technology may be employed in a variety of different applications, including but not limited to human medicine, veterinary medicine, education and inspection. All directional references e.g., upper, lower, inner, outer, upward, downward, left, right, lateral, front, back, top, bottom, above, below; vertical, horizontal, clockwise, counterclockwise, proximal, and distal are only used for identification purposes to aid the reader's understanding of the claimed subject matter, and do not create limitations, particularly as to the position, orientation, or use of the intraluminal imaging system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraluminal sensing device, comprising:
a flexible elongate member comprising a distal portion and a proximal portion and configured to be positioned within a body lumen of a patient;
a sensor configured to obtain physiological data while positioned within the body lumen, wherein the sensor comprises a proximal surface and an opposite, distal surface;
an acoustic matching layer disposed on the distal surface of the sensor; and
a housing positioned at the distal portion of the flexible elongate member and terminating at a distal end, wherein the housing comprises a hollow interior with a planar surface,
wherein the sensor is positioned within the hollow interior of the housing such that the proximal surface of the sensor is disposed on the planar surface of the hollow interior, and
wherein a thickness of the acoustic matching layer is defined by a distance between the distal surface of the sensor and the distal end of the housing.

2. The intraluminal sensing device of claim 1, wherein a distal end of the acoustic matching layer is flush with the distal end of the housing.

3. The intraluminal sensing device of claim 1, wherein the acoustic matching layer comprises an adhesive.

4. The intraluminal sensing device of claim 3, wherein the sensor further comprises a side surface, and wherein the adhesive is disposed on the side surface of the sensor.

5. The intraluminal sensing device of claim 1, wherein the proximal surface of the sensor is planar and the sensor is positioned within the housing such that the proximal surface is parallel with the planar surface along the entire planar surface.

6. The intraluminal sensing device of claim 1, wherein the hollow interior comprises a counterbore, wherein the counterbore comprises a thru-hole extending through the planar surface.

7. The intraluminal sensing device of claim 6, wherein at least a portion of the thru-hole is proximal of the planar surface.

8. The intraluminal sensing device of claim 1, wherein the housing comprises a plurality of layers formed atop one another such that the plurality of layers defines a continuous surface, wherein the hollow interior is defined by the continuous surface.

9. The intraluminal sensing device of claim 1, wherein the sensor further comprises an insulating layer.

10. The intraluminal sensing device of claim 9, wherein the acoustic matching layer is disposed on the insulating layer.

11. The intraluminal sensing device of claim 9, wherein the insulating layer comprises a first material and the acoustic matching layer comprises a different, second material.

12. The intraluminal sensing device of claim 1, wherein the sensor comprises a flow sensor.

13. The intraluminal sensing device of claim 1, further comprising a wire assembly coupled to the sensor and extending through a portion of the hollow interior proximal of the planar surface.

14. The intraluminal sensing device of claim 1, further comprising an adhesive positioned between the sensor and the housing and configured to secure the sensor to the housing.

15. The intraluminal sensing device of claim 14, wherein the adhesive comprises a first material and the acoustic matching layer comprises a different, second material.

16. An intravascular flow-sensing device, comprising:
a guidewire comprising a distal portion and a proximal portion and configured to be positioned within a blood vessel of a patient;
a flow sensor configured to obtain intravascular flow data while positioned within the blood vessel, wherein the flow sensor comprises a proximal surface and an opposite, distal surface;
an acoustic matching layer disposed on a distal surface of the flow sensor; and
a housing positioned at the distal portion of the guidewire and terminating at a distal end, wherein the housing comprises a hollow interior defined by a counterbore, wherein the counterbore comprises a planar surface and a thru-hole,
wherein the flow sensor is positioned within the housing such that the proximal surface of the flow sensor is disposed on the planar surface of the counterbore and a thickness of the acoustic matching layer is defined by a distance between the distal surface of the flow sensor and the distal end of the housing.
